(19) United States
(12) Patent Application Publication
(10) Pub. No.: US 2013/0245024 A1
(43) Pub. Date: Sep. 19, 2013

(54) Combination of PPARy Agonist and a Dipeptidyl Peptidase-Inhibitor for the Treatment of Diabetes and Obesity

(71) Applicants: Dennis Lanfear, Portola Valley, CA (US); Linda Higgins, Palo Alto, CA (US)

(72) Inventors: Dennis Lanfear, Portola Valley, CA (US); Linda Higgins, Palo Alto, CA (US)

(21) Appl. No.: 12/921,058

(22) PCT Filed: Mar. 6, 2009
(86) PCT No.: PCT/US2009/001473

§ 371 Date: Jun. 3, 2013

Related U.S. Application Data
(60) Provisional application No. 61/034,378, filed on Mar. 6, 2008.

(51) Int. Cl. A61K 31/47; A61K 31/40; A61K 31/4985

(52) U.S. Cl. CPC A61K 31/47; A61K 31/4985; A61K 31/40
USPC 514/249; 514/312

(57) ABSTRACT

The present invention relates to pharmaceutical compositions and methods for the treatment of diabetes, obesity or disorders related to diabetes or obesity. The compositions comprise a combination of a particular PPARγ agonist and a particular dipeptidyl peptidase-IV (DPP-IV) inhibitor. The methods include the administration of the combination of a particular PPARγ agonist and a particular dipeptidyl peptidase-IV (DPP-IV) inhibitor.

### 1. FIELD OF THE INVENTION

The present invention relates to the use of a particular PPARγ modulator and a dipeptidyl peptidase-IV (DPP-IV) inhibitor in treating disease, pharmaceutical compositions and kits containing such combinations, and methods of using pharmaceutical compositions comprising both a PPARγ modulator and a DPP-IV inhibitor for the treatment of diabetes, obesity and/or disorders related to diabetes or obesity.

### 2. BACKGROUND OF THE INVENTION

Diabetes is now considered to be a worldwide epidemic. Globally, the number of people with diabetes is expected to rise from the current estimate of 150 millions to 220 million in 2010 and 300 million in 2025. In the United States, it is estimated that as of 2002, 18.2 million people (6.3% of the total population) were diabetic.

Diabetes refers to a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality and thus presents significant public health concerns.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects. However, these patients have developed resistance to the insulin stimulating effect on glucose and lipid metabolism in the main insulin-sensitive tissues.

The clinical-approved treatments for type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic β cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides increase insulin sensitivity resulting in some correction of hyperglycemia. However, the two biguanides, phenformin and metformin, can induce lactic acidosis and nausea/diarrhea. Metformin has fewer side effects than phenformin and is often prescribed for the treatment of Type 2 diabetes.

Peroxisome proliferator-activated receptor γ (“PPARγ”) is one member of the nuclear receptor superfamily of ligand-activated transcription factors and has been shown to be expressed in an adipose tissue-specific manner. Its expression is induced early during the course of differentiation of several preadipocyte cell lines. Additional research has now demonstrated that PPARγ plays a pivotal role in the adipogenic signaling cascade. PPARγ also regulates the ob/leptin gene which is involved in regulating energy homeostasis and adipocyte differentiation, which has been shown to be a critical step to be targeted for anti-obesity and diabetic conditions.

In view of the clinical importance of PPARγ, compounds that modulate PPARγ function can be used for the development of new therapeutic agents for the treatment of diabetes. Potent modulators of PPARγ have been described, for example, in U.S. Pat. No. 6,200,995, U.S. Pat. No. 6,583,157, U.S. Pat. No. 6,653,332, and U.S. Pat. No. 7,041,691. One of these promising modulators, identified as compound 101, is in clinical development for diagnosis or therapeutic treatment of type II diabetes.

Compounds that are inhibitors of the dipeptidyl peptidase-IV (DPP-IV) enzyme are also under investigation as drugs that may be useful in the treatment of diabetes, and particularly type 2 diabetes. The therapeutic utility of DPP-IV inhibitors for the treatment of Type 2 diabetes is discussed for example in (i) D. J. Drucker, 2003, Exp. Opin. Invest. Drugs, 12:87-100; (ii) K. Augustyns, et al., 2003, Exp. Opin. Ther. Patents, 13:499-510; (iii) C. F. Deacon, et al., 2004, Exp. Opin. Investig. Drugs, 13:1091-1102; (iv) A. E. Weber, 2004, J. Med. Chem., 47:4135-4141; (v) J. J. Hoist, 2004, Exp. Opin. Emerg. Drugs, 9: 155-166; (vi) Augustyns et al., 2005, Expert Opinion On Therapeutic Patents, 15(10):1387-1407; (vii) Sebokova et al., 2007, Current Topics in Medicinal Chemistry 7:547-555.

The usefulness of DPP-IV inhibitors in the treatment of type 2 diabetes is based on the fact that DPP-IV in vivo readily inactivates glucagon like peptide-1 (GLP-1) and gastric inhibitory peptide (GIP). GLP-1 and GIP are incretins and are produced when food is consumed. The incretins stimulate production of insulin. Inhibition of DPP-IV leads to decreased inactivation of the incretins, and this in turn results in increased effectiveness of the incretins in stimulating production of insulin by the pancreas. DPP-IV inhibition therefore results in an increased level of serum insulin. Advantageously, since the incretins are produced by the body only when food is consumed, DPP-IV inhibition is not expected to increase the level of insulin at inappropriate times, such as between meals, which can lead to excessively low blood sugar (hypoglycemia). Inhibition of DPP-IV is therefore expected to increase insulin without increasing the risk of hypoglycemia, which is a dangerous side effect associated with the use of insulin secretagogues.

There is a continuing need for new methods of treating diabetes, obesity or disorders related to diabetes or obesity. As described herein, such problems are addressed by providing a combination therapy comprising a particular PPARγ modulator and a DPP-IV inhibitor for the treatment of diabetes, obesity or disorders related to diabetes or obesity.

### 3. SUMMARY OF THE INVENTION

Provided herein are combinations comprising a PPARγ modulator and a DPP-IV inhibitor, which are useful in the treatment of diabetes, obesity, or disorders related to diabetes or obesity. Without being limited to any particular theory or mechanism of action, the combinations are believed to provide one or more clinical advantages over the use of a single agent alone including but not limited to increased clinical efficacy and reduced side effects.

In certain embodiments, the PPARγ modulator of the combinations is compound 101 of formula I or a pharmaceutically acceptable salt, hydrate or polymorph thereof:

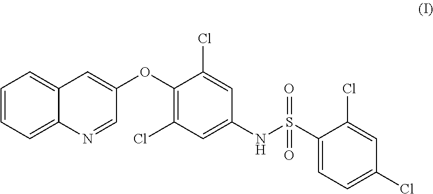 [SMILES: O=S(=O)(Nc1cc(Cl)c(Oc2cnc3ccccc3c2)c(Cl)c1)c1ccc(Cl)cc1Cl]

The DPP-IV inhibitors can be any compound that inhibits the enzymatic activity of DPP-IV. Exemplary DPP-IV inhibitors are described in detail below. In certain embodiments, the DPP-IV inhibitor is selected from the group consisting of vildagliptin, sitagliptin, saxagliptin, PSN9301, SYR 322 and SYR 472.

The combinations are useful in the treatment, of diabetes, obesity, or disorders related to diabetes or obesity. Disorders related to diabetes or obesity are described in detail below. Exemplary disorders related to diabetes or obesity include but are not limited to hyperglycemia, prediabetes, impaired glucose tolerance, impaired fasting glucose, dyslipidemia, hyperlipidemia, hypertriglyceridemia, hypercholesterolemia, low HDL levels, high LDL levels, atherosclerosis, hypertension, sleep apnea, polycystic ovarian syndrome, and metabolic syndrome.

Also provided are methods of treating the above disorders comprising administering to a subject in need thereof a therapeutically effective amount of a compound of formula (I):

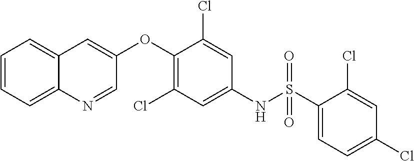 [SMILES: O=S(=O)(Nc1cc(Cl)c(Oc2cnc3ccccc3c2)c(Cl)c1)c1ccc(Cl)cc1Cl]

or a pharmaceutically acceptable salt, hydrate or polymorph thereof; and a therapeutically effective amount of a dipeptidyl peptidase-IV (DPP-IV) inhibitor.

Also provided is a pharmaceutical composition comprising a compound of formula (I):

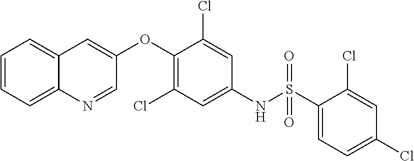 [SMILES: O=S(=O)(Nc1cc(Cl)c(Oc2cnc3ccccc3c2)c(Cl)c1)c1ccc(Cl)cc1Cl]

or a pharmaceutically acceptable salt, hydrate or polymorph thereof; and a therapeutically effective amount of a dipeptidyl peptidase-IV (DPP-IV) inhibitor. Preferably, both agents are provided in a single unit dosage form.

The present invention also provides kits comprising compound 101 or a pharmaceutically acceptable salt, hydrate or polymorph thereof; and a therapeutically effective amount of a DPP-IV inhibitor.

### 4. DETAILED DESCRIPTION OF THE INVENTION

### 4.1 Definitions

The term “composition” as used herein is intended to encompass a product comprising the specified ingredients (and in the specified amounts, if indicated), as well as any product which results, directly or indirectly, from combination of the specified ingredients in the specified amounts.

By “pharmaceutically acceptable” it is meant the diluent; excipient or carrier must be compatible with the other ingredients of the formulation and not deleterious to the recipient thereof.

The term “pharmaceutically acceptable salts” is meant to include salts of active compounds which are prepared with relatively nontoxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like (see, for example, Berge et al. J. Pharm. Sci. 66:1-19 (1977)).

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The terms, “polymorphs” and “polymorphic forms” and related terms herein refer to crystal forms of a molecule. Different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

The term, “solvate,” as used herein, refers to a solid form of a substance which contains solvent. The term “hydrate” refers to a solvate wherein the solvent is water.

The term, “desolvated solvate,” as used herein, refers to a solid form of a substance which can only be made by removing the solvent from a solvate.

The term “alkyl,” as used herein refers to monovalent saturated aliphatic hydrocarbyl groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyl” as defined below.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.”

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

The terms “treat”, “treating” or “treatment”, as used herein, refer to the reduction or amelioration of the progression, severity, and/or duration of a disorder or the eradication, reduction or amelioration of symptoms of a disorder, or the delay of the recurrence or onset of a disorder or one or more symptoms thereof in a subject that results from the administration of one or more compound.

The term “therapeutically effective amount” refers to the amount of the subject salt or polymorph that will elicit the biological or medical response of a tissue, system, animal or human that is being sought by the researcher, veterinarian, medical doctor or other clinician or that is sufficient to prevent development of or alleviate to some extent one or more of the symptoms of the disease being treated.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, “diabetes” refers to type I diabetes mellitus (juvenile diabetes) or type II diabetes mellitus (non-insulin-dependent diabetes mellitus or NIDDM.

The term “obesity” as used herein is a condition in which there is an excess of body fat. In certain embodiments, obesity is defined based on the Body Mass Index (BMI), which is calculated as body weight per height in meters squared (kg/m²). In some embodiments, “obesity” can refer to a condition whereby an otherwise healthy subject has a Body Mass Index (BMI) greater than or equal to 30 kg/m², or a condition whereby a subject with at least one co-morbidity has a BMI greater than or equal to 27 kg/m². In some embodiments, an “obese subject” can be an otherwise healthy subject with a Body Mass Index (BMI) greater than or equal to 30 kg/m² or a subject with at least one co-morbidity with a BMI greater than or equal to 27 kg/m². In some embodiments, an “subject at risk of obesity” can be an otherwise healthy subject with a BMI of 25 kg/m² to less than 30 kg/m² or a subject with at least one co-morbidity with a BMI of 25 kg/m² to less than 27 kg/m².

The term “metabolic syndrome” as used herein is as defined by the Adult Treatment Panel III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. No 01-3670). Briefly, metabolic syndrome occurs when a subject meets three or more of five criteria related to obesity, hypertriglyceridemia, low HDL cholesterol, high blood pressure, and high fasting glucose.

The term “combination,” as used herein in reference to a plurality of pharmaceutically active agents, refers two or more such agents that can be administered in a single unit dosage form or administered in separate dosage forms simultaneously or sequentially.

The term “combination therapy,” as used herein, refers to administration of a combination, as defined above, of pharmaceutically active agents to a subject in need of such administration as described herein.

### 4.2 Combinations Comprising a PPARγ Modulator and a DPP-IV Inhibitor

The present invention provides combinations comprising a particular PPARγ modulator and a DPP-IV inhibitor which are useful in the treatment or prevention of diabetes, obesity, or disorders related to diabetes or obesity. The invention encompasses combinations of agents or methods of employing these agents to treat disease. In one embodiment, these combinations or methods can produce a more effective treatment while reducing or avoiding fewer side effects associated with either treatment alone. Further, the use of the compound 101 and a DPP-IV inhibitor provide a treatment that is more effective than either agent alone. Further, the methods encompass the use of either or both compound 101 and a DPP-IV inhibitor at the same or lower does than those used when either is administered as a single agent.

4.2.1 PPARγ Modulators

The PPARγ modulator used in the combinations of the present invention is (2,4-Dichloro-N-[3,5-dichloro-4-(quinolin-3-yloxy)-phenyl]-benzenesulfonamide benzenesulfonate salt), or compound 101 having the general formula (I), or a pharmaceutically acceptable salt, hydrate or polymorph thereof:

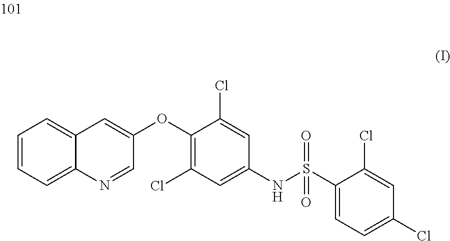

The above PPARγ modulator of compound 101 is disclosed, for example, in international patent publication no. WO 01/00579 (corresponding to U.S. Pat. No. 7,041,691), U.S. Pat. No. 6,200,995, U.S. Pat. No. 6,583,157, U.S. Pat. No. 6,653,332, the contents of which are incorporated by reference in their entireties.

The pharmaceutically acceptable salts and polymorphs of compound 101 are disclosed in U.S. Pat. No. 6,583,157, U.S. Pat. No. 7,041,691, and U.S. Pat. No. 7,223,761, the contents of which are incorporated by reference in their entireties. Each salt of compound 101 can be made from a preparation of compound 101.

The pharmaceutically acceptable salts of compound 101 include but are not limited to benzenesulfonate, hydrochloride salt, or p-toluenesulfonate salt forms of compound 101. Such salt forms are described in detail in U.S. Pat. No. 7,233,761, the contents of which are incorporated by reference in its entirety.

The benzenesulfonate of such salts of formula (I) is according to formula (II):

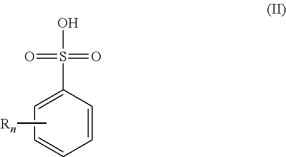

In formula (II), the phenyl ring is optionally substituted with R as described above, and n is any integer from 1 to 5. In certain embodiments, R is heteroalkyl, alkyl or hydrogen, and n is any integer from 1 to 5. In further embodiments, R can be alkyl or hydrogen, and n is any integer from 1 to 5. In some embodiments, R is lower alkyl or hydrogen, and n is any integer from 1 to 5. In some embodiments, each R is hydrogen. The besylate salt of compound 101 is provided by formula (III):

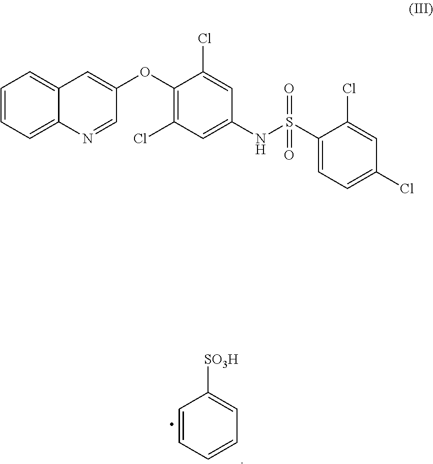 [SMILES: O=S(=O)(Nc1cc(Cl)c(Oc2cnc3ccccc3c2)c(Cl)c1)c1ccc(Cl)cc1Cl ; O=S(=O)(O)c1ccccc1]

Compound 101 can be synthesized or obtained according to any method apparent to those of skill in the art. In some embodiments, compound 101 is prepared according to the methods described in detail in the examples below and in U.S. Pat. Nos. 6,583,157 and 7,041,691, the contents of which are hereby incorporated by reference in their entireties.

Alternatively, compound 101 can be prepared by isolating a salt of compound 101 as described below and converting such a salt of compound 101 to the neutral form by treatment with an appropriate base. For example, compound 101 can be prepared by isolating the hydrochloride salt of compound 101 by filtration, then converting it to the neutral form by treatment with monobasic sodium carbonate in ethyl acetate, or other suitable base. In such embodiments, the hydrochloride salt of compound 101 can be prepared by any method known to one of skill in the art. For example, the hydrochloride salt of compound 101 can be prepared by reacting 3,5-dichloro-4-(quinolin-3-yloxy)-phenylamine with 2,4-dichlorobenzenesulfonylchloride and hydrochloric acid to yield 2,4-dichloro-N-[3,5-dichloro-4-quinolin-3-yloxy)phenyl]-benzenesulfonamide HCl.

Exemplary schemes for the synthesis of compound 101 from 3-hydroxyquinoline are p are described in detail in the examples below. Compound 101 prepared by any method can be contacted with an appropriate acid, either neat or in a suitable inert solvent, to yield the salt forms of the invention. For example, compound 101 can be contacted with an appropriate benzenesulfonic acid to yield the besylate salt forms compound 101.

The polymorphs of compound 101 are described in detail in U.S. Pat. No. 7,233,761, the contents of which is incorporated by reference in its entirety.

Each polymorph of the invention can be made from a preparation of compound 101. Solid compound 101 can be dissolved and then crystallized from the solvent mixtures described below to yield the polymorphic forms of the invention. In particular embodiments of the invention, a besylate salt of compound 101 can be dissolved and then crystallized from the solvent mixtures described below to yield the polymorphic forms of compound 101.

In some embodiments, Form I of a besylate salt of compound 101 (2,4-Dichloro-N-[3,5-dichloro-4-(quinolin-3-yloxy)-phenyl]-benzenesulfonamide benzenesulfonate salt) is used. In such embodiments, the Form I polymorph of the besylate salt of compound 101 may have a melting point of about 180° C. or greater. In a particular embodiment, the Form I polymorph may have a melting point between about 180 and 200° C. When an exemplary Form I polymorph was examined by differential scanning calorimetry according to the methods described in the examples below, it had an endotherm at between about 186.3° C. and about 189.5° C. and an enthalpy of fusion of between about 81.5 J/g and about 89.9 J/g. For example, particular Form I polymorphs of the invention have major X-ray powder diffraction pattern peaks at 7.0, 19.5, 22.0, 24.0, 24.5 and 28° 2θ using Cu Kα radiation. In certain embodiments, the Form I polymorph can have major X-ray powder diffraction pattern peaks at one, two, three, four; five or six of the X-ray powder diffraction pattern peaks at 7.0, 19.5, 22.0, 24.0, 24.5 and 28° 2θ using Cu Kα radiation. In further embodiments, the Form I polymorph can have both a melting point between about 186 and 200° C. and major X-ray powder diffraction pattern peaks at one, two, three, four, five or six of the X-ray powder diffraction pattern peaks at 7.0, 19.5, 22.0, 24.0, 24.5 and 28° 2θ using Cu Kα radiation. In still further embodiments, the Form I polymorph can have major infrared absorbance peaks at one, two, three, four, or five of the infrared absorbance peaks at 1567, 1461, 913, 895, and 881 cm⁻¹.

Form I of the besylate salt of compound 101 can be made by any method of making Form I apparent to those of skill in the art. For example, Form I can be crystallized from ethanol solutions of compound 101 and a hydrate of benzenesulfonic acid. Preferably, an ethanol solution of benzenesulfonic acid hydrate (Aldrich) can be added to solid compound 101 under heat to complete solution; cooling the solution yields Form I. Form I can also be crystallized from solutions of ethyl acetate and ethanol as described in the U.S. Pat. No. 7,233,761, the contents of which is incorporated by reference in its entirety.

In some embodiments, Form II of the besylate salt of compound 101 (2,4-Dichloro-N-[3,5-dichloro-4-(quinolin-3-yloxy)-phenyl]-benzenesulfonamide benzenesulfonate salt) is used. In some embodiments, the Form II polymorph of the besylate salt of compound 101 can have a melting point of about 230° C. or greater. In some embodiments, the Form II polymorph can have a melting point between about 230 and 240° C. An exemplary Form II of the besylate salt of compound 101 displayed surprising stability and had a melting temperature of about 233° C. When an exemplary Form II polymorph was examined by differential scanning calorimetry according to the methods in the examples below, it had an endotherm at about 233.7° C. and an enthalpy of fusion of about 98.9 J/g. For example, particular Form II polymorphs can have major X-ray powder diffraction pattern peaks at 15, 19, 20.5, 23.5, 24.5, 25, 26.5, 29.5 and 30.5° 2θ using Cu Kα radiation. In certain embodiments, the Form II polymorph can have major X-ray powder diffraction pattern peaks at one, two, three, four, five, six, seven or eight of the X-ray powder diffraction pattern peaks at 15, 19, 20.5, 23.5, 24.5, 25, 26.5, 29.5 and 30.5° 2θ using Cu Kα radiation. In certain embodiments, the Form II polymorph of the invention can have both a melting point between about 230 and 240° C. and major X-ray powder diffraction pattern peaks at one, two, three, four, five, six, seven or eight of the X-ray powder diffraction pattern peaks at 15, 19, 20.5, 23.5, 24.5, 25, 26.5, 29.5 and 30.5° 2θ using Cu Kα radiation. In further embodiments, the Form II polymorph can have major infrared absorbance peaks at one, two, three, four, or five of the infrared absorbance peaks at 1573, 1469, 1459, 912, and 859 cm⁻¹.

Form II of the besylate salt of compound 101 can be made by any method apparent to those of skill in the art to make Form II based upon the teachings herein. For example, Form II can be crystallized from solutions of ethyl acetate and ethanol as described in detail in U.S. Pat. No. 7,233,761, the contents of which is incorporated by reference in its entirety. Preferably, Form II of the besylate salt of compound 101 can be prepared by adding an ethanol solution of benzenesulfonic acid to solid compound 101 under heat. The reaction suspension can be stirred under heat, then cooled under further stirring, which yields Form II of the besylate salt of compound 101.

4.2.2 DPP-IV Inhibitors

The DPP-IV inhibitor can be any compound that exhibits inhibition of the enzymatic activity of DPP-IV. In one embodiment, the DPP-IV inhibitor is a compound known to be a DPP-IV inhibitor. In another embodiment, the DPP-IV inhibitor is a selective DPP-IV inhibitor. Examples of DPP-IV inhibitors are described, for example, in (i) D. J. Drucker, 2003, Exp. Opin. Invest. Drugs, 12:87-100; (ii) K. Augustyns, et al., 2003, Exp. Opin. Ther. Patents, 13:499-510; (iii) C. F. Deacon, et al., 2004, Exp. Opin. Investig. Drugs, 13:1091-1102; (iv) A. E. Weber, 2004, J. Med. Chem., 47:4135-4141; (v) J. J. Hoist, 2004, Exp. Opin. Emerg. Drugs, 9: 155-166; (vi) Augustyns et al., 2005, Expert Opinion On Therapeutic Patents, 15(10):1387-1407; (vii) Sebokova et al., 2007, Current Topics in Medicinal Chemistry 7:547-555, the contents of each of which is incorporated by reference in its entirety.

Specific examples of DPP-IV inhibitors include, but are not limited to, dipeptide derivatives or dipeptide mimetics such as alanine-pyrrolidide, isoleucine-thiazolidide, and the pseudosubstrate N-valyl prolyl, O-benzoyl hydroxylamine, as described e.g. in U.S. Pat. Nos. 7,253,172, 7,241,756, 7,238,724, 7,238,720, 7,236,683, 7,235,538, 7,230,074, 7,230,002, 7,229,969, 7,223,573, 7,217,711, 7,208,498, 7,205,409, 7,205,323, 7,196,201, 7,192,952, 7,189,728, 7,186,846, 7,186,731, 7,183,290, 7,183,280, 7,179,809, 7,169,926, 7,169,806, 7,166,579, 7,157,490, 7,144,886, 7,132,443, 7,125,873, 7,125,863, 7,122,555, 7,115,650, 7,109,192, 7,101,871, 7,098,239, 7,084,120, 7,078,397, 7,078,281, 7,074,794, 7,060,722, 7,053,055, 7,034,039, 7,026,316, 6,911,467, 6,890,898, 6,890,905, 6,869,947, 6,867,205, 6,861,440, 6,844,316, 6,849,622, 6,825,169, 6,812,350, 6,803,357, 6,800,650, 6,727,261, 6,716,843, 6,710,040, 6,706,742, 6,699,871, 6,645,995, 6,617,340, 6,699,871, 6,573,287, 6,432,969, 6,395,767, 6,380,398, 6,319,893, 6,303,661, 6,242,422, 6,201,132, 6,172,081, 6,166,063, 6,124,305, 6,110,949, 6,107,317, 6,100,234, 6,040,145, 6,011,155, 5,939,560, 5,462,928, the disclosure of each of which is herein incorporated by reference in its entirety.

Further examples of DPP-IV inhibitors may be found in U.S. Pat. App. Pub. Nos. 20070172525, 20070185061, 2007016750, 20070149451, 20070142383, 20070142436, 20070123579, 20070112059, 20070105890, 20070098781, 20070093492, 20070082932, 20070082908, 20070072810, 20070072804, 20070072803, 20070060547, 20070049619, 20070049596, 20070021477, 20060293297, 20060281796, 20060281727, 20060276487, 20060276410, 20060270722, 20060270701, 20060270679, 20060264457, 20060264433, 20060264401, 20060264400, 20060258646, 20060258621, 20060247226, 20060229286, 20060217428, 20060211682, 20060205711, 20060205675, 20060173056, 20060154866, 20060142585, 20060135767, 20060135561, 20060135512, 20060116393, 20060111336, 20060111428, 20060079541, 20060074058, 20060074087, 20060069116, 20060058323, 20060052382, 20060046978, 20060040963, 20060039974, 20060014953, 20060014764, 20060004074, 20050059724, 20050059716, 20050043292, 20050038020, 20050032804, 20050272765, 20050272652, 20050261271, 20050260732, 20050260712, 20050245538, 20050234235, 20050233978, 20050234108, 20050222242, 20050222222, 20050222140, 20050215784, 20050215603, 20050209249, 20050209159, 20050203095, 20050203031, 20050203027, 20050192324, 20050187227, 20050176771, 20050171093, 20050164989, 20050143377, 20050143405, 20050137224, 20050131019, 20050130985, 20050130981, 20050113310, 20050107390, 20050107309, 20050096348, 20050090539, 20050075330, 20050070719, 20050070706, 20050070535, 20050070531, 20050070530, 20050065148, 20050065145, 20050065144, 20050043299, 20050043292, 20050032804, 20050026921, 20050004205, 20050004117, 20050032804, 20040259903, 20040259902, 20040259883, 20040259870, 20040259843, 20040254226, 20050254167, 20040242898, 20040242636, 20040242568, 20040242566, 20040236102, 20040235752, 20040229926, 20040229848, 20040229820, 20040209891, 20040186153, 20040180925, 20040176428, 20040176406, 20040171555, 20040171848, 20040167341, 20040167133, 20040152745, 20040147434, 20040138215, 20040138214, 20040121964, 20040116328, 20040110817, 20040106656, 20040106802, 20040106655, 20040097510, 20040087587, 20040082570, 20040082497, 20040077645, 20040072892, 20040063935, 20040034014, 20030232788, 20030225102, 20030216450, 20030216382, 20030199528, 20030195188, 20030166578, 20030162820, 20030149071, 20030134802, 20030130281, 20030130199, 20030125304, 20030119750, 20030119738, 20030105077, 20030100563, 20030092630, 20030087950, 20030078247, 20030060494, 20020198242, 20020198205, 20020183367, 20020165164, 20020161001, 20020110560, 20020103384, 20030096857, 20020071838, 20020065239, 20020061839, 20020049164, 20020019411, 20020006899, 20010020006, the disclosure of each of which is herein incorporated by reference in its entirety.

Yet further examples of DPP-IV inhibitors may be found in International Applications WO 07/054,577, WO 07/053,865, WO 05/116029, WO 05/087235, WO 05/082348, WO 05/082849, WO 05/079795, WO 05/075426, WO 05/072530, WO 05/063750, WO 05/058849, WO 05/049022, WO 05/047297, WO 05/044195, WO 05/042488, WO 05/042003, WO 05/040095, WO 05/037828, WO 05/037779, WO 05/034940, WO 05/033099, WO 05/032590, WO 05/030751, WO 05/030127, WO 05/026148, WO 05/025554, WO 05/023762, WO 05/020920, WO 05/19168, WO 05/12312, WO 05/12308, WO 05/12249, WO 05/11581, WO 05/09956, WO 05/03135, WO 05/00848, WO 05/00846, WO 04/112701, WO 04/111051, WO 04/111041, WO 04/110436, WO 04/110375, WO 04/108730, WO 04/104216, WO 04/104215, WO 04/103993, WO 04/103276, WO 04/99134, WO 04/96806, WO 04/92128, WO 04/87650, WO 04/87053, WO 04/85661, WO 04/85378, WO 04/76434, WO 04/76433, WO 04/71454, WO 04/69162, WO 04/67509, WO 04/64778, WO 04/58266, WO 04/52362, WO 04/52850, WO 04/50022, WO 04/50658, WO 04/48379, WO 04/46106, WO 04/43940, WO 04/41820, WO 04/41795, WO 04/37169, WO 04/37181, WO 04/33455, WO 04/32836, WO 04/20407, WO 04/18469, WO 04/18468, WO 04/18467, WO 04/14860, WO 04/09544, WO 04/07468, WO 04/07446, WO 04/04661, WO 04/00327, WO 03/106456, WO 03/104229, WO 03/101958, WO 03/101448, WO 03/99279, WO 03/95425, WO 03/84940, WO 03/82817, WO 03/80633, WO 03/74500, WO 03/72556, WO 03/72528, WO 03/68757, WO 03/68748, WO 03/57666, WO 03/57144, WO 03/55881, WO 03/45228, WO 03/40174, WO 03/38123, WO 03/37327, WO 03/35067, WO 03/35057, WO 03/24965, WO 03/24942, WO 03/22871, WO 03/15775, WO 03/04498, WO 03/04496, WO 03/02530, WO 03/02596, WO 03/02595, WO 03/02593, WO 03/02553, WO 03/02531, WO 03/00181, WO 03/00180, WO 03/00250, WO 02/83109, WO 02/83128, WO 02/76450, WO 02/68420, WO 02/62764, WO 02/55088, WO 02/51836, WO 02/38541, WO 02/34900, WO 02/30891, WO 02/30890, WO 02/14271, WO 02/02560, WO 01/97808, WO 01/96295, WO 01/81337, WO 01/81304, WO 01/68603, WO 01/55105, WO 01/52825, WO 01/34594, WO 00/71135, WO 00/69868, WO 00/56297, WO 00/56296, WO 00/34241, WO 00/23421, WO 00/10549, WO 99/67278, WO 99/62914, WO 99/61431, WO 99/56753, WO 99/25719, WO 99/16864, WO 98/50066, WO 98/50046, WO 98/19998, WO 98/18763, WO 97/40832, WO 95/29691, WO 95/15309, WO 93/10127, WO 93/08259, WO 91/16339, EP 1517907, EP 1513808, EP 1492777, EP 1490335, EP 1489088, EP 1480961, EP 1476435, EP 1476429, EP 1469873, EP 1465891, EP 1463727, EP 1461337, EP 1450794, EP 1446116, EP 1442049, EP 1441719, EP 1426366, EP 1412357, EP1406873, EP 1406872, EP 1406622, EP 1404675, EP 1399420, EP 1399471, EP 1399470, EP 1399469, EP 1399433, EP 1399154, EP 1385508, EP 1377288, EP 1355886, EP 1354882, EP 1338592, EP 1333025, EP 1304327, EP 1301187, EP 1296974, EP 1280797, EP 1282600, EP 1261586, EP 1258476, EP 1254113, EP 1248604, EP 1245568, EP 1215207, EP 1228061, EP 1137635, EP 1123272, EP 1104293, EP 1082314, EP 1050540, EP 1043328, EP 0995440, EP 0980249, EP 0975359, EP 0731789, EP 0641347, EP 0610317, EP 0528858, CA 2466870, CA 2433090, CA 2339537, CA 2289125, CA 2289124, CA 2123128, DD 296075, DE 19834591, DE 19828113, DE 19823831, DE 19616486, DE 10333935, DE 10327439, DE 10256264, DE 10251927, DE 10238477, DE 10238470, DE 10238243, DE 10143840, FR 2824825, FR 2822826, JP2005507261; JP 2005505531, JP 2005502624, JP 2005500321, JP 2005500308, JP2005023038, JP 2004536115, JP 2004535445, JP 2004535433, JP 2004534836, JP 2004534815, JP 2004532220, JP 2004530729, JP 2004525929, JP 2004525179, JP 2004522786, JP 2004521149, JP 2004503531, JP 2004315496, JP 2004244412, JP 2004043429, JP 2004035574, JP 2004026820, JP 2004026678, JP 2004002368, JP 2004002367, JP 2003535898, JP 2003535034, JP 2003531204, JP 2003531191, JP 2003531118, JP 2003524591, JP 2003520849, JP 2003327532, JP 2003300977, JP 2003238566, JP 2002531547, JP 2002527504, JP 2002517401, JP 2002516318, JP 2002363157, JP 2002356472, JP 2002356471, JP 2002265439, JP 2001510442, JP 2000511559, JP 2000327689, JP 2000191616, JP 1998182613, JP 1998081666, JP 1997509921, JP 1995501078, JP 1993508624, the disclosure of each of which is herein incorporated by reference in its entirety.

In certain embodiments, the DPP-IV inhibitor is a small molecule with molecular weight less than 1000, 700 or 500 Daltons.

In certain embodiments, the DPP-IV inhibitor is a β-aminoacid derivative, such as 3(R)-Amino-1-[3-(trifluoromethyl)-5,6,7,8-tetrahydro[1,2,4]triazolo[4,3-a-]pyrazin-7-yl]-4-(2,4,5-trifluorophenyl)butan-1-one (MK-0431), or its pharmaceutical salt, hydrate or polymorph, which are described in detail in U.S. Pat. No. 6,699,871, EP 1412357, WO 03/04498, and US 2003100563, the disclosure of each of which is herein incorporated by reference in its entirety. In some embodiments, the DPP-IV inhibitor is sitagliptin. Sitagliptin is described as an orally active and selective DPP-IV inhibitor and was recently approved for the treatment of diabetes alone or in combination with metformin or a PPARγ agonist. See U.S. Pat. No. 6,699,871, Kim et al., 2005, J. Med. Chem. 48:141-151, the contents of each of which is herein incorporated by reference in its entirety.

In certain embodiments, the DPP-IV inhibitor is cyanopyrrolidide, such as (1-[[3-hydroxy-1-adamantyl)amino]acetyl]-2-cyano-(S)-pyrrolidine (LAF237 or vildagliptin), 1-[2-[5-cyanopyridin-2-yl)amino]ethylamino]acetyl-2-cyano-(S)-pyrrolidine (NVP-DPP728), or (1S,3S,5S)-2-[2(S)-Amino-2-(3-hydroxyadamantan-1-yl)acetyl]-2-azabicyclo[-3.1.0]hexane-3-carbonitrile (saxagliptin or BMS-47718), which are disclosed in detail, for example, in U.S. Pat. Nos. 6,617,340, 6,432,969, 6,395,767, 6,166,063, 6,124,305, 6,110,949, 6,011,155, 6,107,317, WO 98/19998 and JP 2000511559, WO 00/34241, EP 1137635, and JP 2002531547, the contents of each of which is herein incorporated by reference in its entirety.

In some embodiments, the DPP-IV inhibitor is vildagliptin. In some embodiments, the DPP-IV inhibitor is NVP-DPP728. Vildagliptin and NVP-DPP728 are described as an orally active and selective DPP-IV inhibitor. See Villhauer et al, 2002, J Med Chem 45:2362-2365, Villhauer et al, 2003, J Med Chem 46:2774-2789, the contents of each of which are incorporated by reference in its entirety. Vildagliptin (LAF 237) is currently undergoing Phase III clinical trial in the United States.

In certain embodiments, the DPP-IV inhibitor is saxagliptin. Saxagliptin is currently in Phase III clinical trail in the U.S. and Europe for the treatment of type II diabetes. See Augeri et al., 2005, J. Med. Chem. 48(5):5025-5037, the contents of which is incorporated by reference in its entirety.

In certain embodiments, the DPP-IV inhibitor is 3-(L-Isoleucyl)thiazolidine (isoleucine-thiazolidide or PSN-9301). Isoleucine-thiazolidide may be found in JP 2001510442, WO 97/40832, U.S. Pat. No. 6,303,661, and DE 19616486, the disclosure of each of which is herein incorporated by reference in its entirety. Isoleucine-thiazolidide is described as an orally active and selective DPP-IV inhibitor. See Pederson et al, 1998, Diabetes 47:1253-1258; Epstein et al., 2007, Curr. Opion. Investig. Drugs, 8(4):331-337, the contents of which is herein incorporated by reference in their entireties.

In certain embodiments, the DPP-IV inhibitor is SYR-322 or SYR-472 such as described in U.S. Pat. Nos. 7,169,926 and 7,034,039, the contents of which in herein incorporated by reference in their entireties.

In certain embodiments, the DPP-IV inhibitor is valine-pyrrolidide, such as disclosed in Deacon et al, Diabetes (1998) 47:764769; the contents of which is herein incorporated by reference in its entirety.

In certain embodiments, the DPP-IV inhibitor is [1-[2(S)-Amino-3-methylbutyryl]pyrrolidin-2(R)-yl]boronic acid (PT-100).

In certain embodiments, the DPP-IV inhibitor is β-phenethylamine, such as described in Nordhoff et al., 2006, Bioorganic Medical Chemistry Letters 16:1744-1748, the contents of which is incorporated by reference in its entirety.

In certain embodiments, the DPP-IV inhibitor is PT-630 (DB-160), such as described in Application Publication No. WO 06/034435, which is herein incorporated by reference in its entirety.

In certain embodiments, the DPP-IV inhibitor is ABT-341, such as described in Pei et al., J. Med. Chem. 2006 Nov. 2; 49(22):6439-42, which is herein incorporated by reference in its entirety.

In certain embodiments, the DPP-IV inhibitor is ABT-279, such as described in Madar et al., J. Med. Chem. 2006 Oct. 19; 49(21):6416-20, which is herein incorporated by reference in its entirety.

In certain embodiments, the DPP-IV inhibitor is BI-1356/Ondero, such as described in Application Publication No. WO 04/18468, which is herein incorporated by reference in its entirety.

In certain embodiments, the DPP-IV inhibitor is SYR-619.

In certain embodiments, the DPP-IV inhibitor is GSK-823093.

In certain embodiments, the DPP-IV inhibitor is PSN 9301.

In certain embodiments, the DPP-IV inhibitor is TA-6666.

In certain embodiments, the DPP-IV inhibitor is CR-14023.

In certain embodiments, the DPP-IV inhibitor is CR-14025.

In certain embodiments, the DPP-IV inhibitor is CR-14240.

In certain embodiments, the DPP-IV inhibitor is CR-13651.

In certain embodiments, the DPP-IV inhibitor is NNC-72-2138.

In certain embodiments, the DPP-IV inhibitor is NN-7201.

In certain embodiments, the DPP-IV inhibitor is PHX-1149.

In certain embodiments, the DPP-IV inhibitor is PHX-1004.

In certain embodiments, the DPP-IV inhibitor is SNT-189379.

In certain embodiments, the DPP-IV inhibitor is GRC-8087.

In certain embodiments, the DPP-IV inhibitor is PT-630.

In certain embodiments, the DPP-IV inhibitor is SK-0403.

In certain embodiments, the DPP-IV inhibitor is GSK-825964.

In certain embodiments, the DPP-IV inhibitor is TS-021.

In certain embodiments, the DPP-IV inhibitor is GRC-8200.

In certain embodiments, the DPP-IV inhibitor is GRC-8116.

In certain embodiments, the DPP-IV inhibitor is FE107542.

In certain embodiments, the DPP-IV inhibitor is MP-513.

In certain embodiments, the DPP-IV inhibitor is BI356.

In certain embodiments, the DPP-IV inhibitor is ALS 2-0426.

In certain embodiments, the DPP-IV inhibitor is ABT279.

In certain embodiments, the DPP-IV inhibitor is TS-201.

In certain embodiments, the DPP-IV inhibitor is KRP-104.

In certain embodiments, the DPP-IV inhibitor is R1579.

In certain embodiments, the DPP-IV inhibitor is LY2463665.

In certain embodiments, the DPP-IV inhibitor is ARI-2243.

In certain embodiments, the DPP-IV inhibitor is SSR-162369.

In certain embodiments, the DPP-IV inhibitor is not valine-pyrrolidide. In certain embodiments, the DPP-IV inhibitor is not alanine-pyrrolidide. In certain embodiments, the DPP-IV inhibitor is not 3-(L-Isoleucyl)thiazolidine (isoleucine-thiazolidide). In certain embodiments, the DPP-IV inhibitor is not N-valyl propyl,O-benzoyl hydroxylamine. In certain embodiments, the DPP-IV inhibitor is not 1-[2-[5-cyanopyridin-2-yl)amino]ethylamino]acetyl-2-cyano-(S)-pyrrolidine (NVP-DPP728). In certain embodiments, the DPP-IV inhibitor is not 3(R)-Amino-1-[3-(trifluoromethyl)-5,6,7,8-tetrahydro[1,2,4]triazolo[4,3-a-]pyrazin-7-yl]-4-(2,4,5-trifluorophenyl)butan-1-one (MK-0431). In certain embodiments, the DPP-IV inhibitor is not (1-[[3-hydroxy-1-adamantyl)amino]acetyl]-2-cyano-(S)-pyrrolidine (LAF237 or vildagliptin). In certain embodiments, the DPP-IV inhibitor is not (1S,3S,5S)-2-[2(S)-Amino-2-(3-hydroxyadamantan-1-yl)acetyl]-2-azabicyclo[3.1.0]hexane-3-carbonitrile (BMS-477118). In certain embodiments, the DPP-IV inhibitor is not [1-[2(S)-Amino-3-methylbutyryl]pyrrolidin-2(R)-yl]boronic acid (PT-100). In certain embodiments, the DPP-IV inhibitor is not GSK-823093. In certain embodiments, the DPP-IV inhibitor is not PSN-9301. In certain embodiments, the DPP-IV inhibitor is not TA-6666. In certain embodiments, the DPP-IV inhibitor is not SYR-322. In certain embodiments, the DPP-IV inhibitor is not SYR-619. In certain embodiments, the DPP-IV inhibitor is not CR-14023. In certain embodiments, the DPP-IV inhibitor is not CR-14025. In certain embodiments, the DPP-IV inhibitor is not CR-14240. In certain embodiments, the DPP-IV inhibitor is not CR-13651. In certain embodiments, the DPP-IV inhibitor is not NNC-72-2138. In certain embodiments, the DPP-IV inhibitor is not NN-7201. In certain embodiments, the DPP-IV inhibitor is not PHX-1149. In certain embodiments, the DPP-IV inhibitor is not PHX-1004. In certain embodiments, the DPP-IV inhibitor is not SNT-189379. In certain embodiments, the DPP-IV inhibitor is not GRC-8087. In certain embodiments, the DPP-IV inhibitor is not PT-630. In certain embodiments, the DPP-IV inhibitor is not SK-0403. In certain embodiments, the DPP-IV inhibitor is not GSK-825964. In certain embodiments, the DPP-IV inhibitor is not TS-021. In certain embodiments, the DPP-IV inhibitor is not GRC-8200. In certain embodiments, the DPP-IV inhibitor is not GRC-8116. In certain embodiments, the DPP-IV inhibitor is not FE107542.

In certain embodiments, the DPP-IV inhibitor is not MP-513.

In certain embodiments, the DPP-IV inhibitor is not BI356.

In certain embodiments, the DPP-IV inhibitor is not ALS 2-0426.

In certain embodiments, the DPP-IV inhibitor is not ABT279.

In certain embodiments, the DPP-IV inhibitor is not TS-201.

In certain embodiments, the DPP-IV inhibitor is not KRP-104.

In certain embodiments, the DPP-IV inhibitor is not R1579.

In certain embodiments, the DPP-IV inhibitor is not LY2463665.

In certain embodiments, the DPP-IV inhibitor is not ARI-2243.

In certain embodiments, the DPP-IV inhibitor is not SSR-162369.

In certain embodiments, the DPP-IV inhibitor is selected from the group consisting of vildagliptin, sitagliptin, saxagliptin, PSN9301, SYR 322 and SYR 472.

4.2.3 Pharmaceutical Compositions

The combinations of the present invention may be in the form of a pharmaceutical composition that further comprises a pharmaceutically acceptable diluent, excipient or carrier.

The pharmaceutical compositions may conveniently be presented in unit dosage form and may be prepared by any of the methods well known in the art of pharmacy. In general, the pharmaceutical compositions are prepared by uniformly and intimately bringing the combinations into association with a liquid carrier or a finely divided solid carrier or both, and then, if necessary, shaping the product into the desired formulation. In the pharmaceutical composition, each of the active ingredient is included in an amount sufficient to produce the desired effect upon the process, condition or disease to be modulated, prevented, or treated.

The pharmaceutical compositions containing the combinations may be in a form suitable for oral use, for example, as tablets, troches, lozenges, aqueous or oily suspensions, dispersible powders or granules, emulsions, hard or soft capsules, or syrups, solutions, or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the combinations in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452, and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the combinations in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the combinations in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions may also be administered in the form of suppositories suitable for rectal administration of the drug. These compositions can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

For topical use, creams, ointments, jellies, solutions or suspensions, etc., containing the salts or polymorphs of the present invention may be employed. As used herein, topical application is also meant to include the use of mouth washes and gargles.

The pharmaceutical compositions may further comprise other therapeutically active compounds known to one skilled in the art to be useful in the treatment or prevention of the above mentioned pathological conditions.

### 4.3 Methods of Treatment Using a PPARγ Modulator and a DPP-IV Inhibitor

The present invention provides the use of a particular PPARγ modulator or a pharmaceutically acceptable salt, hydrate, or polymorph thereof, and a DPP-IV inhibitor or a pharmaceutically acceptable salt, hydrate, or polymorph thereof as a combination therapy for the treatment of various disorders such as diabetes, obesity, disorders related to diabetes or obesity.

Thus, the present invention provides methods of treating diabetes, obesity or disorders related to diabetes or obesity by administering to a subject having diabetes, obesity, or a related disorder, a therapeutically effective amount of a PPARγ modulator, e.g., a compound of formula (I):

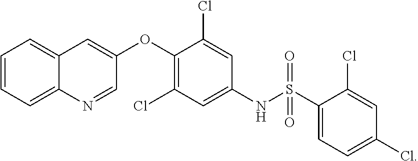 [SMILES: O=S(=O)(Nc1cc(Cl)c(Oc2cnc3ccccc3c2)c(Cl)c1)c1ccc(Cl)cc1Cl]

or a pharmaceutically acceptable salt, hydrate or polymorph thereof; and a therapeutically effective amount of a dipeptidyl peptidase-IV (DPP-IV) inhibitor. The administrations may be simultaneous or sequential.

The subject can be an animal such as, for example, a mammal, including, but not limited to, a primate (e.g., a human), a cow, a sheep, a goat, a horse, a dog, a cat, a rabbit, a rat, a mouse and the like. In certain embodiments, the subject is human.

The combinations of the present invention are useful for the treatment of diabetes. The diabetes may be due to any cause, whether genetic or environmental.

Diabetes treatable with the compositions of the present invention, includes both insulin-dependent diabetes mellitus (i.e., IDDM, also known as Type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. The development of Type 2 diabetes is related to obesity; most of the Type 2 diabetics are also obese. The combinations of the present invention are useful for treating both Type 1 and Type 2 diabetes. The combinations are believed to be especially effective for treating Type 2 diabetes. The combinations of the present invention are also useful for treating and/or preventing gestational diabetes mellitus.

In some embodiments, diabetes can be characterized by a fasting plasma glucose level of greater than or equal to 126 mg/dl. In some embodiments, a diabetic subject can have a fasting plasma glucose level of greater than or equal to 126 mg/dl. In some embodiments, prediabetes can be characterized by an impaired fasting plasma glucose (FPG) level of greater than or equal to 110 mg/dl and less than 126 mg/dl; or impaired glucose tolerance; or insulin resistance. In some embodiments, a prediabetic subject can be a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

The combinations of the present invention are useful for the treatment of obesity. The term “obesity” as used herein is a condition in which there is an excess of body fat. In some embodiments, obesity can be defined based on the Body Mass Index (BMI), which is calculated as body weight per height in meters squared (kg/m²). In some embodiments, obesity can refer to a condition whereby an otherwise healthy subject has a Body Mass Index (BMI) greater than or equal to 30 kg/m², or a condition whereby a subject with at least one co-morbidity has a BMI greater than or equal to 27 kg/m². In some embodiments, an obese subject can be an otherwise healthy subject with a Body Mass Index (BMI) greater than or equal to 30 kg/m² or a subject with at least one co-morbidity with a BMI greater than or equal to 27 kg/m².

The combinations of the present invention are useful for the treatment of disorders related to diabetes or obesity. In certain embodiments, the disorders related to diabetes or obesity is selected from the group consisting of hyperglycemia, prediabetes, impaired glucose tolerance, impaired fasting glucose, dyslipidemia, hyperlipidemia, hypertriglyceridemia, hypercholesterolemia, low HDL levels, high LDL levels, atherosclerosis, hypertension, sleep apnea, polycystic ovarian syndrome, and metabolic syndrome.

The diabetes-related disorders herein can be any disorders associated with, caused by, or result from diabetes. Examples of diabetes-related disorders include but are not limited to hyperglycemia, impaired glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, irritable bowel syndrome, inflammatory bowel disease, including Crohn's disease and ulcerative colitis, other inflammatory conditions, pancreatitis, abdominal obesity, neurodegenerative disease, retinopathy, neoplastic conditions, adipose cell tumors, adipose cell carcinomas, such as liposarcoma, prostate cancer and other cancers, including gastric, breast, bladder and colon cancers, angiogenesis, Alzheimer's disease, psoriasis, high blood pressure, Metabolic Syndrome, ovarian hyperandrogenism (polycystic ovary syndrome), and other disorders where insulin resistance is a component, such as sleep apnea. The combinations of the present invention are particularly useful for the treatment of hyperglycemia, impaired glucose tolerance, obesity, dyslipidemia, hyperlipidemia, hypertriglyceridemia, hypercholesterolemia, low HDL levels, atherosclerosis, and metabolic syndrome.

The combinations of the present invention are useful for the treatment of obesity-related disorders. The obesity-related disorders herein can be any disorders associated with, caused by, or result from obesity. Examples of obesity-related disorders include but are not limited to obesity, diabetes, overeating, binge eating, and bulimia, hypertension, elevated plasma insulin concentrations and insulin resistance, dyslipidemia, hyperlipidemia, endometrial, breast, prostate, kidney and colon cancer, osteoarthritis, obstructive sleep apnea, gallstones, heart disease, abnormal heart rhythms and arrythmias, myocardial infarction, congestive heart failure, coronary heart disease, sudden death, stroke, polycystic ovary disease, craniopharyngioma, Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. Further examples of obesity-related disorders are metabolic syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, lower back pain, gallbladder disease, hyperuricemia, gout, and kidney cancer, and increased anesthetic risk. The combinations of the present invention are also useful to treat Alzheimer's disease.

The combinations of the present invention are also useful for the treatment or prevention of metabolic syndrome. “Metabolic syndrome” can be defined as described in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following disorders: abdominal obesity, hypertriglyceridemia, low HDL cholesterol, high blood pressure, and high fasting plasma glucose. The criteria for these are defined in ATP-III. Treatment of metabolic syndrome refers to the administration of the combinations of the present invention to a subject with metabolic syndrome or a subject that has developed two of the disorders that define metabolic syndrome, but has not yet developed three or more of the disorders that define metabolic syndrome.

4.3.1 Routes of Administration and Dosage

The particular PPARγ modulator and the DPP-IV inhibitor can be administered in a single pharmaceutical dosage formulation which contains the particular PPARγ modulator and the DPP-IV inhibitor, or be administered in its own separate pharmaceutical dosage formulation. Where separate dosage formulations are used, the individual active ingredient can be administered at essentially the same time, i.e., concurrently, or at separately staggered times, i.e. sequentially prior to or subsequent to the administration of the other active ingredient. The instant methods are therefore to be understood to include all such regimes of simultaneous or non-simultaneous treatment.

The methods further encompass continuous and cyclic administration of one or both pharmaceutically active agents. Thus, in certain embodiments, one agent can be administered continuously, e.g., daily, while the other is administered cyclically, e.g., weekly or monthly or daily for a week and then not administered for three weeks, followed by another similar cycle. In other embodiments, both agents are administered cyclically or continuously.

Administration in these various ways are suitable for the present compositions as long as the beneficial pharmaceutical effect of the combination of the particular PPARγ modulator and the DPP-IV inhibitor is realized by the subject at substantially the same time. Such beneficial effect is preferably achieved when the target blood level concentrations of each active ingredient are maintained at substantially the same time. It is preferred that the combination of the particular PPARγ modulator and the DPP-IV inhibitor be co-administered concurrently on a once-a-day dosing schedule; however, varying dosing schedules, such as twice or more times per day, is also encompassed herein. A single oral dosage formulation comprised of both active ingredient in the combination is preferred. A single dosage formulation will provide convenience for the patient, which is an important consideration especially for patients with diabetes, metabolic syndrome, or obese patients who may be in need of multiple medications.

Pharmaceutical compositions of the particular PPARγ modulator and DPP-IV inhibitor, either individually or in combination, may be prepared by methods well known in the art as described above, e.g., by means of conventional mixing, dissolving, granulation, dragee-making, levitating, emulsifying, encapsulating, entrapping, lyophilizing processes or spray drying.

The dosage of the particular PPARγ modulator and the DPP-IV inhibitor can be determined in accordance with the judgment of one of skill in the art. In the case where an single composition is employed, a suitable dosage range can be, e.g. from about 0.001 mg/kg to about 100 mg/kg of each compound in the composition per day, preferably from about 0.01 mg to about 2000 mg per day. For oral administration, the compositions can be provided in the form of tablets containing from 0.01 mg to 1,000 mg, e.g. 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850, 1,000 and 2,000 milligrams of each compound for the symptomatic adjustment of the dosage to the subject to be treated. This dosage regimen may be adjusted to provide the optimal therapeutic response.

The particular PPARγ modulator in the combinations of the present invention can be administered at a daily dosage of from about 0.1 mg to about 100 mg per kilogram of animal body weight, preferably given as a single daily dose or in divided doses two to six times a day, or in sustained release form. For human, the total daily dosage can be from about 1.0 mg to about 1000 mg, preferably from about 10 mg to about 200 mg. In the case of a 70 kg adult human, the total daily dose can be generally be from about 5 mg to about 350 mg. This dosage regimen may be adjusted to provide the optimal therapeutic response.

The DPP-IV inhibitors in the combinations of the present invention can be administered at a daily dosage of from about 0.1 mg to about 100 mg per kilogram of animal body weight, preferably given as a single daily dose or in divided doses two to six times a day, or in sustained release form. For human, the total daily dosage can be from about 1.0 mg to about 1000 mg, preferably from about 10 mg to about 200 mg. In the case of a 70 kg adult human, the total daily dose can generally be from about 5 mg to about 350 mg. This dosage regimen may be adjusted to provide the optimal therapeutic response.

The weight ratio of the particular PPARγ modulator and the DPP-IV inhibitor may be varied and will depend upon the effective dose of each ingredient. Generally, an effective dose of each will be used.

Depending on the disease to be treated and the subject's condition, the particular PPARγ modulator and the DPP-IV inhibitor may be administered by oral, parenteral (e.g., intramuscular, intraperitoneal, intravenous, ICV, intracisternal injection or infusion, subcutaneous injection, or implant), inhalation spray, nasal, vaginal, rectal, sublingual, or topical routes of administration and may be formulated, alone or together, in suitable dosage unit formulations containing conventional non-toxic pharmaceutically acceptable diluents, excipients or carriers appropriate for each route of administration. When the particular PPARγ modulator and the DPP-IV inhibitor are administered separately, they may be administered by different routes.

It will be understood, however, that the specific dose level and frequency of dosage for any particular patient may be varied and will depend upon a variety of factors including the activity of the specific agent employed, the metabolic stability and length of action of that agent, the age, body weight, general health, sex, diet, mode and time of administration, rate of excretion, drug combination, the severity of the particular condition, and the host undergoing therapy.

4.3.2 Use of Further Active Agents

The combinations of the present invention can be further combined with other compounds having related utilities to treat diabetes, obesity or disorders related to diabetes or obesity. Thus, in certain embodiments, it is contemplated that three or more pharmaceutically active agents can be used in the combinations and methods described herein. In many instances, administration of the subject compounds or compositions in conjunction with these alternative agents enhances the efficacy of such agents. Accordingly, in some instances, the present combinations, when combined or administered in combination with, e.g., additional anti-diabetic agents, can be used in dosages which are less than the expected amounts when used alone, or less than the calculated amounts for combination therapy.

For example, suitable agents for combination therapy include those that are currently commercially available and those that are in development or will be developed. Exemplary agents useful in the treatment of diabetes, obesity or disorders related to diabetes or obesity include, but are not limited to: (a) anti-diabetic agents such as insulin, sulfonylureas (e.g., meglinatide, tolbutamide, chlorpropamide, acetohexamide, tolazamide, glyburide, glipizide and glimepiride), biguanides, e.g., metformin (Glucophage®), α-glucosidase inhibitors (acarbose), thiazolidinone compounds, e.g., rosiglitazone (Avandia®, troglitazone (Rezulin®) and pioglitazone (Actos®); (b) β₃ adrenergic receptor agonists, leptin or derivatives thereof and neuropeptide Y antagonists; (c) bile acid sequestrants (e.g., cholestyramine and colestipol), HMG-CoA reductase inhibitors, e.g., statins (e.g., lovastatin, atorvastatin, fluvastatin, pravastatin and simvastatin), nicotinic acid (niacin), fibric acid derivatives (e.g., gemfibrozil and clofibrate) and nitroglycerin.

In certain embodiments, the combination comprises a compound of formula (I), a DPP-IV inhibitor as described herein, and metformin.

### 4.4 Kits

The present invention further provides kits comprising a particular PPARγ modulator and a DPP-IV inhibitor. The particular PPARγ modulator and the DPP-IV inhibitors are described in detail in section 5.2 above.

In certain embodiments, the kits comprise a therapeutically effective amount of a compound of formula (I):

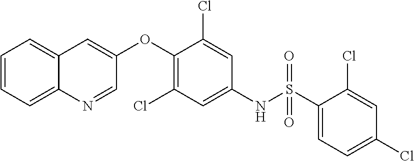 [SMILES: O=S(=O)(Nc1cc(Cl)c(Oc2cnc3ccccc3c2)c(Cl)c1)c1ccc(Cl)cc1Cl]

or a pharmaceutically acceptable salt, hydrate or polymorph thereof; and a therapeutically effective amount of a dipeptidyl peptidase-IV (DPP-IV) inhibitor.

In certain embodiments, the kits comprise two separate pharmaceutical compositions: a first unit dosage form comprising a therapeutically effective amount of a particular PPARγ modulator, i.e. compound 101 of a pharmaceutically acceptable salt, hydrate, or polymorph thereof, and a pharmaceutically acceptable carrier or diluent in a first unit dosage form, and a second unit dosage form comprising a therapeutically effective amount of a DPP-IV inhibitor, or a pharmaceutically acceptable salt, hydrate, or polymorph thereof, and a pharmaceutically acceptable carrier or diluent in a second unit dosage form.

In some embodiments, the kits further comprises a container. Such kits are especially suited for the delivery of solid oral forms such as tablets or capsules. Such kits may include a number of unit dosages. Such kits can include a card having the dosages oriented in the order of their intended use. An example of such a kit is a “blister pack.” Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

In some embodiments, the kits further comprise a label or labeling with instruction for using the kits. For example, the label of labeling can provide dosage information and specific methods of administration for the particular PPARγ modulator and the DPP-IV inhibitor.

### 5. EXAMPLES

### 5.1 Example 1

### Synthesis of Compound 101

This example provides an exemplary synthesis of compound 101. Alternate methods of synthesizing compound 101, including methods of synthesizing acid addition salts of compound 101 are described below; still other alternate synthetic methods will be apparent to those of skill in the art.

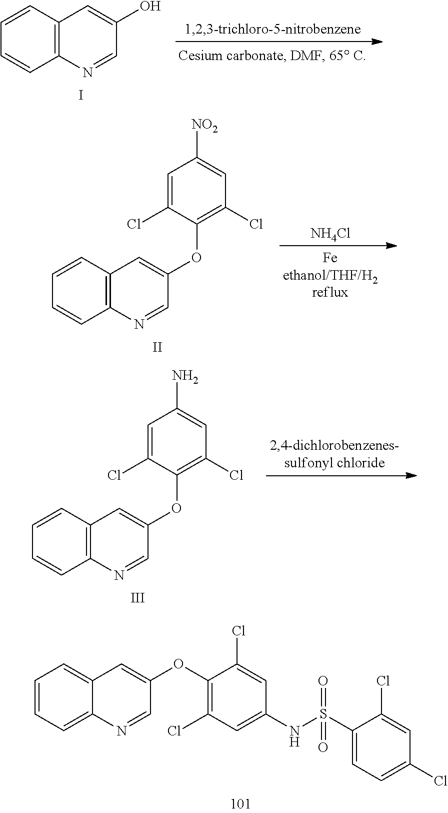

### 3-(2,6-Dichloro-4-nitro-phenoxy)-3,4-dihydro-quinoline (II)

3-Hydroxyquinoline (I) (prepared according to the procedure of Naumann et. al., Synthesis 4:279-281 (1990)) (3 g) and 1,2,3-trichloro-5-nitrobenzene (4.7 g) were dissolved in DMF (80 mL) and heated with cesium carbonate (7.4 g) for 2 h at 60° C. The reaction was poured into ice/water (500 mL). The resulting off-white precipitate was collected by filtration and rinsed with hexane to afford compound II as a solid (6.9 g) suitable for use in the next reaction.

¹H NMR in CDCl₃ δ 8.863 (d, J=2.2 Hz, 1H), 8.360 (s, 2H), 8.106 (d, J=8.6 Hz, 1H), 7.646 (m, 2H), 7.529 (d, J=8.6 Hz, 1H), 7.160 (d, J=2.2 Hz, 1H).

### 3,5-Dichloro-4-(3,4-dihydro-quinolin-3-yloxy)-phenylamine (III)

To a solution of compound II (6.9 g) in ethanol/THF/water (ratio 40:20:10) was added ammonium chloride (3.3 g) and powdered iron (3.4 g). This mixture was heated to reflux for 5 h. The hot mixture was then filtered through Celite and concentrated. The residue was dissolved in ethyl acetate and washed with saturated NaHCO₃ solution followed by water and then brine. The solution was dried over magnesium sulfate and concentrated to afford compound III as an off-white solid (5.6 g).

¹H NMR in (DMSO) δ 8.846 (d, J=2.9 Hz, 1H), 8.010 (m, 1H), 7.915 (m, 1H), 7.645 (m, 1H), 7.560 (m, 1H), 7.401 (d, J=2.9 Hz, 1H), 6.778 (s, 2H), 5.762 (s, 2H).

### 2,4-Dichloro-N-[3,5-dichloro-4-(quinolin-3-yloxy)-phenyl]-benzenesulfonamide (101)

Treatment of the aniline III with 2,4-dichlorobenzenesulfonyl chloride according to conventional methods gave compound 101

¹H NMR (d₆-acetone) δ 9.9 (1H, br s), 8.794 (1H, d, J=2.9 Hz), 8.23 (1H, d, J=8.4 Hz), 8.035 (1H, br d, J=8.4 Hz), 7.793 (1H, d, J=1.5 Hz), 7.78 (1H, m), 7.62-7.70 (2H, m), 7.57 (1H, td, J=6.8, 1.2 Hz), 7.476 (2H, s), 7.364 (1H, d, J=2.6 Hz). MS (M-H) 511.0.

### 5.2 Example 2

### PPARγ Ligand Binding

Using methods similar to Lehmann et al., J. Biol. Chem. 270:12953-12956 (1995), compound 101, prepared according to Example 1, exhibited an IC₅₀ of less than 1 μM in a PPARγ ligand binding assay utilizing [³H]-BRL 49653 as the radioligand.

### 5.3 Example 3

### Preparation of the Besylate Salt of Compound 101

The besylate salt of compound 101 was synthesized from 2,4-dichloro-N-[3,5-dichloro-4-quinolin-3-yloxy)phenyl]-benzenesulfonamide HCl prepared as described in detail in U.S. Pat. No. 7,223,671, the contents of which is incorporated by reference in its entirety. The hydrochloride salt 2,4-dichloro-N-[3,5-dichloro-4-quinolin-3-yloxy)phenyl]-benzenesulfonamide HCl was converted to the besylate salt, via the free base, using a sodium bicarbonate/ethyl acetate biphasic reaction solution. Separation of the organic layer followed by solvent exchange with ethanol precipitated the besylate salt (6) of compound 101 in 84% yield. Starting from 4-aminoquinoline (2), the overall yield of the besylate salt (6) of compound 101 was 73%.

### 5.4 Example 4

### Oral Dosage Forms Comprising Compound 101 and Sitagliptin

This example provides oral dosage forms comprising compound 101 and sitagliptin.

Compound 101 can be synthesized as described in Examples above. Sitagliptin can be synthesized as described in detail, for example, in U.S. Pat. No. 6,699,871 and U.S. Pat. App. Pub. No. 2006/027022, the contents of which are incorporated by reference in their entireties.

Oral dosage forms of enterostatin may contain 10, 25, 50, 75 or 100 mg stigliptin and 0.5, 1, 2, 3, 4, 5., 7.5 or 10 mg compound 101. They may comprise excipients or non-hygroscopic additives. Suitable excipients may be starches, sugars, and micro-crystalline cellulose etc. Suitable non-hygroscopic may be dibasic calcium phosphate anhydrous, calcium sulfate, powdered cellulose, dextrose and lactitol etc. Oral dosage forms of enterostatin may be in the form of tablets or capsules.

Exemplary oral dosage forms include:

Composition 101:

0.5, 1, 2, 3, 5, 10 or 25 mg compound 101, 50 mg sitagliptin, 50 mg, microcrytalline cellulose, 10 mg hydroxyprophylcellulose, 10 mg croscarmellose sodium, 5 mg magnesium sterate.

Composition 102:

0.5, 1, 2, 3, 5, 10 or 25 mg compound 101, 25 mg sitagliptin, 50 mg, microcrytalline cellulose, 10 mg hydroxyprophylcellulose, 10 mg croscarmellose sodium, 5 mg magnesium sterate.

Composition 103:

0.5, 1, 2, 3, 5, 10 or 25 mg compound 101, 25 mg sitagliptin, 60 mg, microcrytalline cellulose, 15 mg hydroxyprophylcellulose, 15 mg croscarmellose sodium, 10 mg magnesium sterate.

### 5.5 Example 5

### Combination Therapy Using Compound 101 and Sitagliptin

This example illustrates combination therapy of compound 101 and sitagliptin by oral administration.

Patients having NIDDM (Type 2 diabetes mellitus) are selected for therapy. The patients weigh between 70-100 kilograms.

Compound 101 is orally administered in a dosage of 0.1 to 1,000 milligrams twice daily, more typically 0.5, 1, 2, 3, 5, 10 or 25 mg daily or twice daily. Sitagliptin is orally administered in a dosage of 20 to 100 mg once per day. For infants or children the doses suggested are lowered in a linear fashion based on body weight or surface area.

One third of the patients are orally administered sitagliptin daily. One third of the patients are orally administered compound 101 daily. The remaining third of the patient population is administered sitagliptin daily as well as compound 101. The patients are monitored for improvement in the manifestations of the disease and for side effects, such as body weight gain and signs of liver toxicity.

### 5.6 Example 6

### Combination Therapy Using Compound 101 and Vildagliptin

This example illustrates combination therapy of compound 101 and vildagliptin by oral administration.

Patients having NIDDM (Type 2 diabetes mellitus) are selected for therapy. The patients weigh between 70-100 kilograms.

Compound 101 is orally administered in a dosage of 0.1 to 1,000 milligrams twice daily, more typically 0.5, 1, 2, 3, 5, 10 or 25 mg daily or twice daily. Vildagliptin is orally administered in a dosage of 20 to 100 mg once per day. For infants or children the doses suggested are lowered in a linear fashion based on body weight or surface area.

One third of the patients are orally administered vildagliptin daily. One third of the patients are orally administered compound 101 daily. The remaining third of the patient population is administered vildagliptin daily as well as compound 101. The patients are monitored for improvement in the manifestations of the disease and for side effects, such as body weight gain and signs of liver toxicity.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A pharmaceutical composition comprising a compound of formula (I): 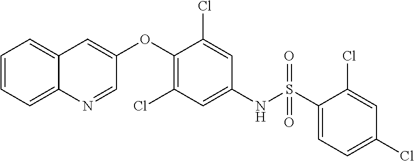 [SMILES: O=S(=O)(Nc1cc(Cl)c(Oc2cnc3ccccc3c2)c(Cl)c1)c1ccc(Cl)cc1Cl]
or a pharmaceutically acceptable salt, hydrate or polymorph thereof; and a dipeptidyl peptidase-IV (DPP-IV) inhibitor.

2. The pharmaceutical composition of claim 1, wherein the pharmaceutically acceptable salt of the compound is a benzenesulfonate salt, a hydrochloride salt or a p-toluenesulfonate salt of the compound.

3. The pharmaceutical composition of claim 1, wherein the DPP-IV inhibitor is selected from the group consisting of vildagliptin, sitagliptin, saxagliptin, PSN9301, SYR 322 and SYR 472.

4. The pharmaceutical composition of claim 1, wherein the pharmaceutical composition further comprises a pharmaceutically acceptable diluent, excipient or carrier.

5. The pharmaceutical composition of claim 1, wherein the composition comprises 0.5 mg of the compound of formula (I).

6. The pharmaceutical composition of claim 1, wherein the composition comprises 1 mg of compound of formula (I).

7. The pharmaceutical composition of claim 1, wherein the composition comprises 2 mg of compound of formula (I).

8. The pharmaceutical composition of claim 1, wherein the composition comprises 3 mg of compound of formula (I).

9. The pharmaceutical composition of claim 1, wherein the composition comprises 4 mg of compound of formula (I).

10. The pharmaceutical composition of claim 1, wherein the composition comprises 5 mg of compound of formula (I).

11. A method of treating a condition selected from the group consisting of diabetes, obesity, or a disorder related to diabetes or obesity, comprising administering to a subject in need thereof a therapeutically effective amount of a compound of formula (I): 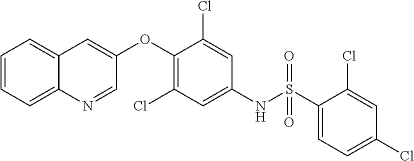 [SMILES: O=S(=O)(Nc1cc(Cl)c(Oc2cnc3ccccc3c2)c(Cl)c1)c1ccc(Cl)cc1Cl]
or a pharmaceutically acceptable salt, hydrate or polymorph thereof; and a therapeutically effective amount of a dipeptidyl peptidase-IV (DPP-IV) inhibitor.

12. The method of claim 11 wherein said subject is human.

13. The method of claim 11 wherein said condition is diabetes.

14. The method of claim 11 wherein said condition is obesity.

15. The method of claim 11 wherein said disorder related to diabetes or obesity is selected from the group consisting of hyperglycemia, prediabetes, impaired glucose tolerance, impaired fasting glucose, dyslipidemia, hyperlipidemia, hypertriglyceridemia, hypercholesterolemia, low HDL levels, high LDL levels, atherosclerosis, hypertension, sleep apnea, polycystic ovarian syndrome, and Metabolic Syndrome.

16. The method of claim 11, wherein the pharmaceutically acceptable salt of the compound is a benzenesulfonate salt, a hydrochloride salt or a p-toluenesulfonate salt of the compound.

17. The method of claim 11, wherein the DPP-IV inhibitor is selected from the group consisting ofvildagliptin, sitagliptin, saxagliptin, PSN-9301, SYR 322 and SYR 472.

18-19. (canceled)

20. The method of claim 11, wherein said compound and said DPP-IV inhibitor are administered together in a single pharmaceutical composition.

21. The method of claim 11, wherein said compound and said DPP-IV inhibitor are administered together in a separate pharmaceutical composition.

22. The method of claim 11, wherein said compound and said DPP-IV inhibitor are administered concurrently.

23-26. (canceled)
